(19) United States
(12) Patent Application Publication
(10) Pub. No.: US 2026/0283501 A1
(43) Pub. Date: Sep. 24, 2026

(54) Systems and Methods for Automated Detection of Breath Sounds

(71) Applicant: The Board of Trustees of the Leland Stanford Junior University, Stanford, CA (US)

(72) Inventors: Matthew Muffly, Stanford, CA (US); Christopher David Chafe, Woodside, CA (US); Matthew Wright, Stanford, CA (US); Steven Michael Herron, Stanford, CA (US)

(73) Assignee: The Board of Trustees of the Leland Stanford Junior University, Stanford, CA (US)

(21) Appl. No.: 19/168,277

(22) PCT Filed: Apr. 5, 2024
(86) PCT No.: PCT/US2024/023428

§ 371 Date: Sep. 23, 2025

Related U.S. Application Data
(60) Provisional application No. 63/494,431, filed on Apr. 5, 2023.

(51) Int. Cl. A61B 5/08; A61B 5/00; A61B 7/00; A61B 7/04

(52) U.S. Cl. CPC A61B 5/0826; A61B 5/0823; A61B 5/7203; A61B 5/725; A61B 5/7257; A61B 5/7264; A61B 5/746; A61B 7/003; A61B 7/04; A61B 5/6831; A61B 2562/0204; A61B 2562/14; A61B 2562/16; H04R 2410/05

(57) ABSTRACT

Systems and methods for automated detection of breath sounds in accordance with embodiments of the invention are illustrated. One embodiment includes a respiration monitoring system, comprising a listening device, comprising a first microphone embedded in a gel matrix, where the gel matrix includes a first channel between a surface of the gel matrix and the first microphone, and a processing unit, comprising a processor, and a memory, where the memory contains a respiration monitoring application that configures the processor to obtain a first audio signal from the first microphone, where the first audio signal includes breath sounds recorded at the skin of a patient, generate a spectrogram of the first audio signal, provide the spectrogram to a breath identification model, receive an identified breathing condition from the breath identification mode, and provide the identified breathing condition.

### CROSS-REFERENCE TO RELATED APPLICATIONS

The current application claims the benefit of and priority under 35 U.S.C. § 119(e) to U.S. Provisional Patent Application No. 63/494,431 entitled “Systems and Methods for Automated Detection of Breath Sounds” filed Apr. 5, 2023. The disclosure of U.S. Provisional Patent Application No. 63/494,431 is hereby incorporated by reference in its entirety for all purposes.

### FIELD OF THE INVENTION

The present invention generally relates to automatic detection of breath sounds, and more specifically to providing patient specific medical alerts based on breathing.

### BACKGROUND

The human respiratory system is a vital and continuously functioning organ system that handles oxygen intake and carbon dioxide disposal via breathing. Listening to patient breathing has been a diagnostic modality for many years. Indeed, an iconic tool of the medical profession is the stethoscope and enables the detection of impaired, or absent, respiratory airflow. Respiratory distress can be a sign of illness, or even immediate danger.

Machine learning is a computer science field concerning development of models that can learn from data and perform tasks without explicit instructions. Neural networks are a class of machine learning model that utilize layers of nodes (or neurons) to modify an input signal. Convolutional neural networks (CNNs) are a class of neural network that include at least one convolution layer. CNNs are particularly useful for image classification.

### SUMMARY OF THE INVENTION

Systems and methods for automated detection of breath sounds in accordance with embodiments of the invention are illustrated. One embodiment includes a respiration monitoring system, comprising a listening device, comprising a first microphone embedded in a gel matrix, where the gel matrix includes a first channel between a surface of the gel matrix and the first microphone, and a processing unit, comprising a processor, and a memory, where the memory contains a respiration monitoring application that configures the processor to obtain a first audio signal from the first microphone, where the first audio signal includes breath sounds recorded at the skin of a patient, generate a spectrogram of the first audio signal, provide the spectrogram to a breath identification model, receive an identified breathing condition from the breath identification mode, and provide the identified breathing condition.

In a further embodiment, the listening device further includes a second microphone embedded in the gel matrix, where the gel matrix further includes a second channel between the surface of the gel matrix and the second microphone, and where the first microphone and second microphone face opposite directions, and the respiration monitoring application further configures the processor to obtain a second audio signal from the second microphone, where the second audio signal includes audio signals from an environment of the patient, phase invert the second audio signal, apply noise cancellation to the first audio signal using the phase inverted second audio signal, and generate the spectrogram using the noise cancelled first audio signal.

In still another embodiment, the first and second channels are filled with a material having a lower density than the gel matrix that allows sounds in the acoustic range of breathing to pass efficiently.

In a still further embodiment, the method further includes steps for a shell that covers a portion of the gel matrix over the second microphone.

In yet another embodiment, to generate the spectrogram, the respiration monitoring application further directs the processor to generate a linear magnitude vector using a Fourier transform.

In a yet further embodiment, the listening device and the processing unit communicate wirelessly.

In another additional embodiment, the method further includes steps for a telemetry unit configured to receive the first audio signal and provide the first audio signal to the processing unit.

In a further additional embodiment, the method further includes steps for a warning device configured to provide an alarm in response to an abnormal identified breathing condition.

In another embodiment again, the breath identification model is a convolutional neural network.

In a further embodiment again, the respiration monitoring application further directs the processor to filter the first audio signal below 250 Hz and above 1500 Hz.

One embodiment includes a respiration monitoring method, comprising obtaining, at a processing unit, a first audio signal from a first microphone, where the first audio signal includes breath sounds recorded at the skin of a patient, the first microphone embedded in a gel matrix, and where the gel matrix includes a first channel between a surface of the gel matrix and the first microphone, generating a spectrogram of the first audio signal, providing the spectrogram to a breath identification model, receiving an identified breathing condition from the breath identification mode, and providing the identified breathing condition.

In still yet another embodiment, the method further includes steps for obtaining a second audio signal from a second microphone at the processing unit, where the second audio signal includes audio signals from an environment of the patient, the second microphone is embedded in the gel matrix, the gel matrix further includes a second channel between the surface of the gel matrix and the second microphone, and the first microphone and second microphone face opposite directions, phase inverting the second audio signal, applying noise cancellation to the first audio signal using the phase inverted second audio signal, and generating the spectrogram using the noise cancelled first audio signal.

In a still yet further embodiment, the first and second channels are filled with a material having a lower density than the gel matrix that allows sounds in the acoustic range of breathing to pass efficiently.

In still another additional embodiment, the gel matrix is covered by a shell that over a portion of the gel matrix over the second microphone.

In a still further additional embodiment, generating the spectrogram includes generating a linear magnitude vector using a Fourier transform.

In still another embodiment again, the first microphone and the processing unit communicate wirelessly.

In a still further embodiment again, the first microphone provides the first audio signal to a telemetry unit, which in turn provides the first audio signal to the processing unit.

In yet another additional embodiment, further including providing a warning using a warning device in response to an abnormal identified breathing condition.

In a yet further additional embodiment, the breath identification model is a convolutional neural network.

In yet another embodiment again, the method further includes steps for filtering the first audio signal below 250 Hz and above 1500 Hz.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIGS. 1A and 1B illustrate a top view and a side view of a listening device, respectively, in accordance with an embodiment of the invention.

FIG. 2 illustrates a listening device with a housing in accordance with an embodiment of the invention.

FIGS. 3A and 3B illustrate a top view and a side view of an attachment device, respectively, in accordance with an embodiment of the invention.

FIG. 4 is a system architecture for a respiration monitoring system in accordance with an embodiment of the invention.

FIG. 5 is another system architecture for a respiration monitoring system in accordance with an embodiment of the invention.

FIG. 6 is a block diagram of a processing unit in accordance with an embodiment of the invention.

FIG. 7 is a flow chart for a respiration monitoring process in accordance with an embodiment of the invention.

FIGS. 8A-M illustrate spectrograms of various breathing problems recorded using a respiration monitoring system in accordance with an embodiment of the invention.

### DETAILED DESCRIPTION

Patients in hospitals, especially when sedated for procedures or surgery, are at significant risk for respiratory-related issues including respiratory obstruction. Moreover, when a breathing tube is removed (e.g. after surgery in the operating room), there is often no direct monitoring of respiratory airflow. Currently, monitoring devices for measuring oxygen saturation of the hemoglobin in the blood (SpO2) are used as part of patient telemetry monitoring using, for example, a pulse oximeter. However, SpO2 is a late indicator of effective respiratory airflow, with a lag time of up to 2 minutes. Respiratory rate can be monitored remotely as well using automated sensors like those that measure thoracic impedance. However, respiratory rate, even in conjunction with SpO2 can fail to accurately alert medical practitioners to many forms of labored or obstructed breathing. Medical staff therefore often perform intermittent “spot checks” of breathing to confirm adequate respiratory airflow in patients (even those connected to telemetry) to try and solve these issues, and to provide a low-cost, high accuracy respiration monitoring solution generally.

Respiration monitoring systems and methods described herein utilize acoustic sensors for monitoring breath sounds and can automatically identify changes in respiratory airflow quality from baseline, such as respiratory airflow obstruction. In turn, medical staff can be immediately alerted to patient breathing troubles at their onset. A wireless microphone can be used to record sounds for processing on a computing platform. In some embodiments, the wireless microphone is positioned with a housing in order to amplify audio signals associated with breath sounds.

In numerous embodiments, a listening device with one or more microphones (e.g. Micro-Electromechanical Systems (MEMS)) in direct apposition with or embedded in a matrix of material approximating the acoustic impedance of human tissue (e.g. gelatin, silicone, polyacrylamide, polymethylpentene, agarose, alginate, polydimethylsiloxane, polyvinyl alcohol, polyethylene glycol diacrylate, etc.) is used. In some embodiments, at least the material matrix and microphone are encased within a housing. In many embodiments, the housing and/or material matrix has a channel between at least one microphone and the exterior of the device. In some embodiments, the channel is air filled. In other embodiments, the channel is filled with a lower density matrix that allows sounds in the acoustic range of breathing to pass efficiently. The device can be attached to the neck of a patient and detects at least breath sounds at the neck. In a variety of embodiments, the device is placed in close proximity to the trachea. These sounds are processed to filter ambient sound contamination and subsequently processed by a machine learning model to identify regular breathing, irregular breathing, and/or respiratory airflow quality. In numerous embodiments, the machine learning model identifies a specific type of irregularity in breathing or respiratory airflow quality.

Wireless functionality, while not required, can be helpful in transmitting data to a wide variety of displays such as (but not limited to) smartphones, tablets, computers, clinical monitors, etc. without the requisite panoply of wire adapters, and overall unobtrusiveness. In some embodiments, the listening device is connected (either via wired or wireless connection) to a telemetry unit to incorporate transmission into existing hospital networks, or to a controller for subsequent processing.

In numerous embodiments, a computing module (similar to an IntelliVue MMX Multi-Measurement Module such as those produced by Koninklijke Philips N.V. of Amsterdam, Netherlands) can be added to a clinical computing tower. The computing module can contain a wireless receiver for receiving data from the listening device and can direct connected displays to provide alerts and other patient status updates. However, as can readily be appreciated, in numerous embodiments, a general purpose (or other purpose-built) computing platform can be used without departing from the scope or spirit of the invention. A discussion of hardware components is found below, followed by a discussion of audio processing to identify breath sounds.

### Respiration Monitoring Systems

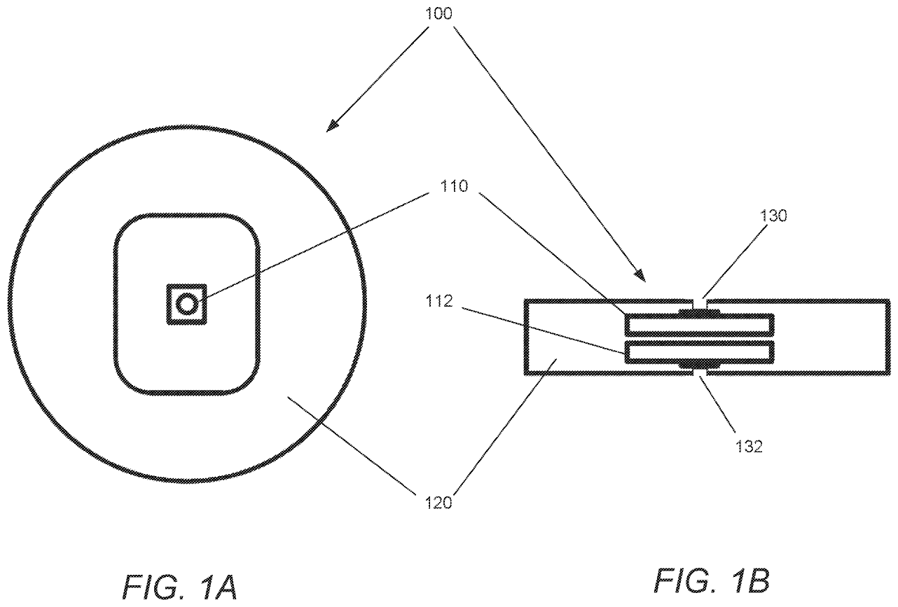

Respiration monitoring systems utilize specialized listening devices to record breathing sounds and process the sounds to identify irregular breathing or respiratory airflow patterns in patients. In numerous embodiments, respiration monitoring systems are remote monitoring devices configured to alert medical professionals and caretakers immediately when breathing and respiratory airflow issues are identified. Turning now to FIGS. 1A and 1B, a listening device component of a respiration monitoring system in accordance with an embodiment of the invention is illustrated. The listening device 100 includes two opposing microphones 110 and 112 that are embedded in a silicone gel 120. In many embodiments, the silicone gel is replaced with a different gel material that has similar acoustic impedance. In a variety of embodiments, the acoustic sensor of the microphones are not covered by the gel. In various embodiments, the microphones are embedded within the gel such that an open channel from the acoustic sensor to the surface is formed. Listening device 100 has two channels 130 and 132. While a flat disc is illustrated in FIGS. 1A and 1B, other shapes such as (but not limited to) squares, triangles, and/or any other shape molded to lay flat against the skin can be used as appropriate to the requirements of specific applications of embodiments of the invention. In numerous embodiments, more than two microphones are used. For example, an array of microphones can be used. In various embodiments, an array of microphones may all point a single direction to in order to increase signal-to-noise ratio. In many embodiments, the gel has a diameter of approximately 20 mm, and a width of approximately 5 mm. However, these numbers can be reduced (or increased) depending on the dimensions of the microphones used (e.g. between 10 mm and 30 mm, and between 2 mm and 8mm). As can readily be appreciated, the size of the microphone is not critical so long as it can record breath sounds and environment sounds without causing significant discomfort to the patient.

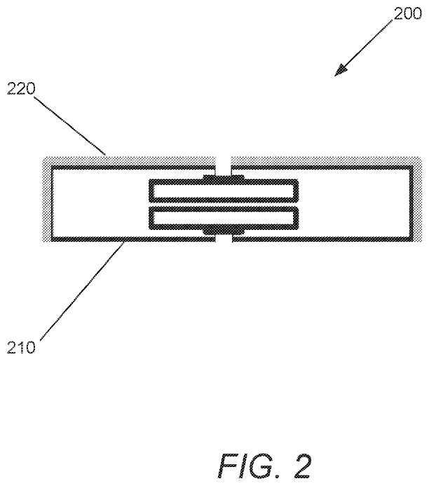

The assembly of the microphones and gel can be housed within an outer shell. Turning now to FIG. 2, a listening device with housing in accordance with an embodiment of the invention is illustrated. The gel and microphone assembly 210 is housed within a shell 220. In numerous embodiments, the shell only covers the exterior (outward facing with respect to the body) portion of the assembly. This way, the silicon gel is exposed directly to the skin of the patient when used. The shell 220 includes a channel aligned with the acoustic sensor component of the outward facing microphone such that the channel is exposed to air.

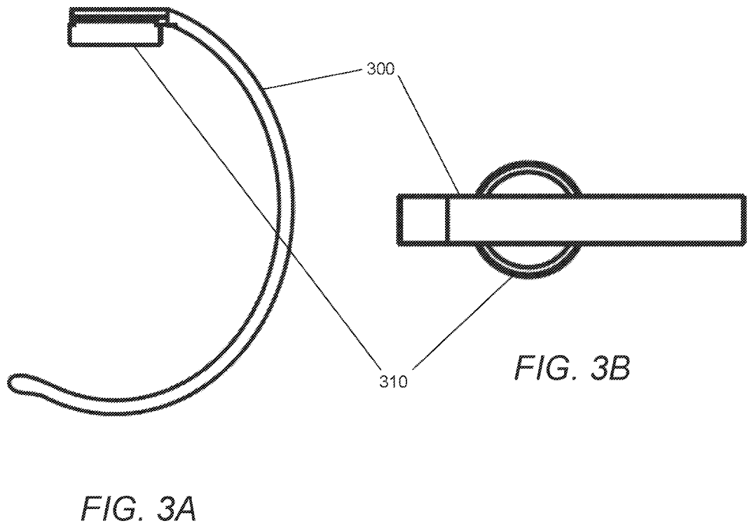

In many embodiments, the shell is utilized as a hard attachment point for a attachment device. keeps the listening device against the patient's skin in a static position. Turning now to FIGS. 3A and 3B, an example attachment device that is designed to keep the listening device against a patient's neck in accordance with an embodiment of the invention is illustrated. Attachment device 300 holds listening device 310. As can be readily appreciated, any number of different attachment devices can be used, including (but not limited to) straps, tapes, and any other hardware component as appropriate to the requirements of specific applications of embodiments of the invention. Depending on the attachment location, different attachment devices can be used. For example, a modification to the processing can be made in order to enable detection of other biological sounds (e.g. heart sounds) by retraining the classification model. Different attachment locations on the body may be more appropriate for different applications, and therefore different attachment devices may be preferred. Further, shells are not required, and in numerous embodiments, the assembly is attached without a shell. For example, adhesive may be used to attach the assembly like a sticker to the patient's neck.

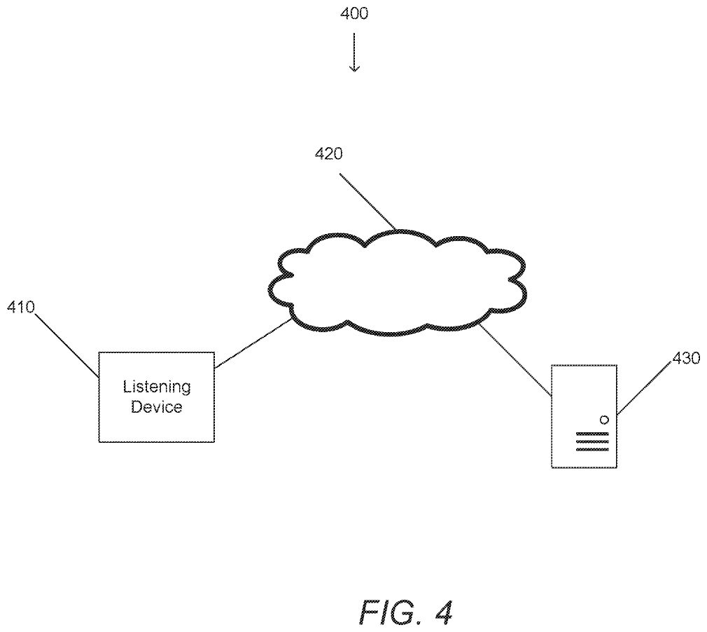

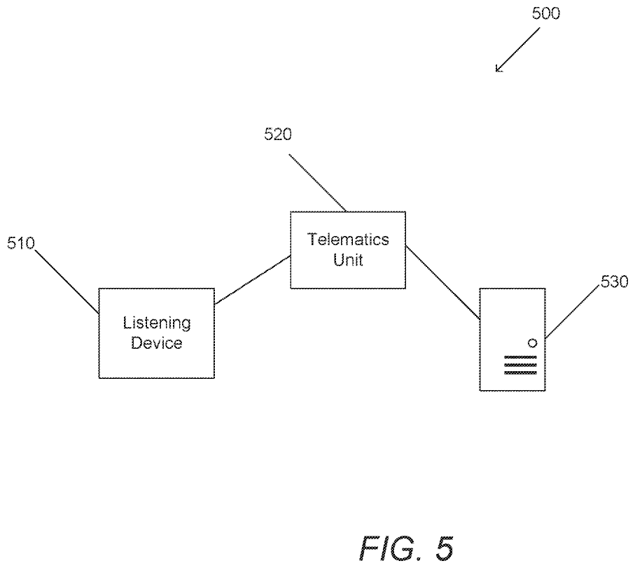

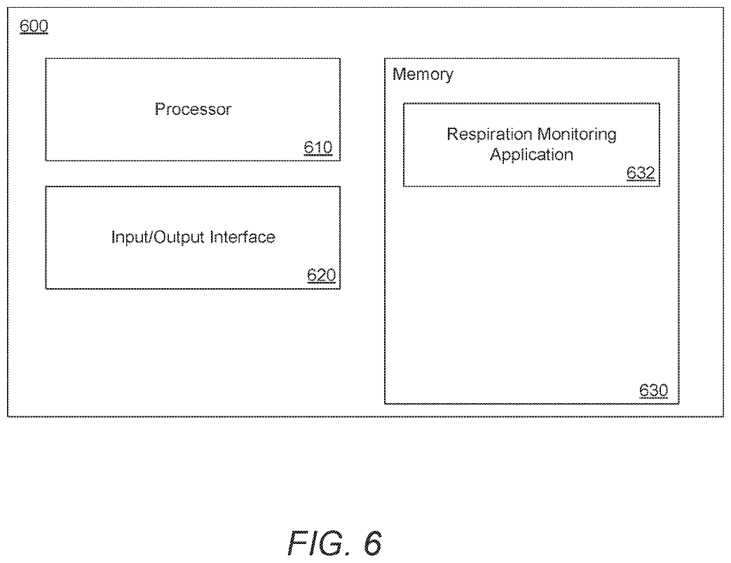

Turning now to FIG. 4, a system architecture for a respiration monitoring system in accordance with an embodiment of the invention is illustrated. System 400 includes a listening device 410. In many embodiments, the listening device is as described above, and is positioned against the neck of a patient. The listening device records audio signals from both the internal facing and external facing microphones. The audio signals are transmitted via a network 520 to a processing unit 520. In many embodiments, the processing unit is a general-purpose computer, however the processing unit may be a medical computer or any other device with sufficient compute programmed to perform respiration monitoring as described herein. In numerous embodiments, the listening device has an integrated processing component which can perform some or all of the processes a processing unit performs in system 200.

Another system architecture in accordance with an embodiment of the invention is illustrated in FIG. 5. Here, system 500 connects listening device 510 to a telematics unit 520, which in turn wirelessly transmits data to the processing unit 530. Telematics units are commonly found in medical settings and are specialized devices for transmitting recorded signals from various medical sensors and devices to a central monitoring area. In some embodiments, wired connections are used instead of wireless connections. Indeed, some listening devices may use wireless communication to transmit recorded audio signals instead of using a wired connector. As can be readily appreciated, any number of different system architectures that acquire and process data from listening devices can be utilized as appropriate to the requirements of specific applications of embodiments of the invention.

FIG. 6 is a block diagram for a processing unit in accordance with an embodiment of the invention. Processing unit 600 includes a processor 610. In many embodiments, the processor is a central processing unit. However, processors can be any number of different types of logic processing circuitry including (but not limited to), graphics processing units, application-specific integrated circuits, field-programmable gate arrays, and/or any other circuit capable of carrying out various processing tasks described herein. Processing unit 600 further includes an input/output (I/O) interface 620. The I/O interface is capable of receiving and/or transmitting data via any number of different communication modalities as appropriate to the requirements of specific applications of embodiments of the invention. In many embodiments, the I/O interface may communicate via wired and/or wireless communication to connected devices including (but not limited to) listening devices, displays, other computing systems, and/or any other device depending on system architecture. I/O interface 620 is communicatively coupled to at least processor 600.

The processing unit 600 further includes a memory 630. Memory 630 can be made of volatile memory, nonvolatile memory, or any combination thereof. Memory 630 is communicatively coupled to at least the processor 610. Memory 630 stores a respiration monitoring application 632. Respiration monitoring applications are a set of instructions stored within the machine-readable medium of the memory that configure the processor to carry out respiration monitoring processes as described herein. As can readily be appreciated, other system architectures can be used as appropriate to the requirements of specific applications of embodiments of the invention, including those that utilize general purpose computing hardware.

While specific system architectures are described above, any number of different computing architectures can be used in combination with listening devices in order to obtain recorded audio signals and process them without departing from the scope or spirit of the invention. For example, while microphones are discussed above, any acoustic signal recording device can be used. Further, in some embodiments, a substrate is not used around the microphone (or recording device). Respiration monitoring processes are discussed in further detail below.

### Respiration Monitoring Processes

Respiration monitoring processes involve the recording of audio signals from a patient using listening devices, and performing various computational steps in order to yield a near-real time identification of breathing and respiratory airflow problems a patient may be experiencing at any given moment. In numerous embodiments, identification of a breathing problem triggers an alert which can be provided to medical professionals in order to provide immediate intervention as necessary. Listening devices as described herein produce two separate audio signals: an inward facing signal and an outward signal. The inward signal is the “breath audio signal” which is obtained by recording against the skin of the patient's neck. By merits of the silicone gel and the corresponding inward facing channel through the gel, a degree of audio isolation is provided. However, the audio isolation is often not perfect. The outward signal is recorded from the opposing microphone, which by necessity faces the environment of the patient (e.g. the hospital room). The mirrored nature of the microphones and channels provides a similar acoustic recording profile. The outward signal, referred to as the “environment audio signal” is used to cancel out environmental, non-breathing sounds from the breath audio signal. In numerous embodiments, both signals can be filtered prior to noise cancelling. In many embodiments, the signals are band pass filtered to remove signal outside of approximately 250 Hz to 1500 Hz, which is the range of most human breathing sounds. Further, the recorded signals are converted from the analog domain to the digital domain for processing. The cancelled signal can then be processed by a machine learning model to identify any breathing issues.

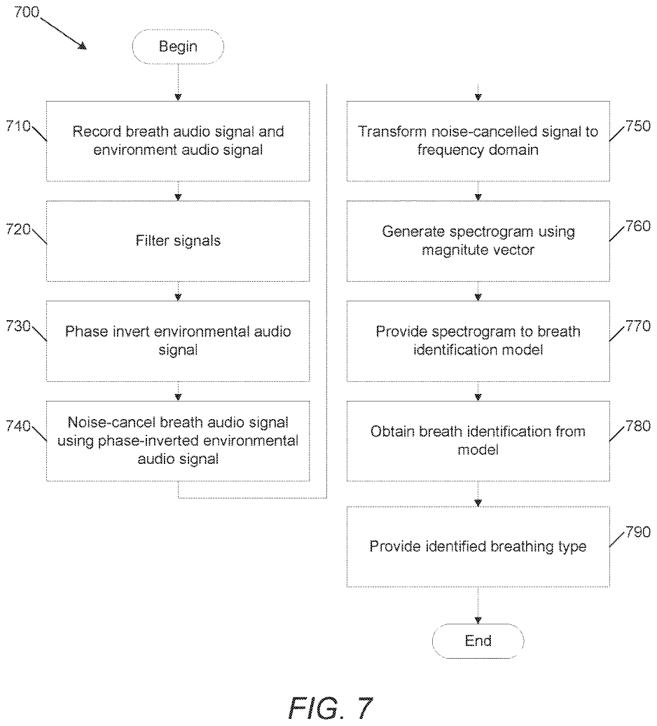

Turning now to FIG. 7, a flow chart for a respiration monitoring process in accordance with an embodiment of the invention is illustrated. Process 700 includes recording (710) the breath audio signal and the environment audio signal using the listening device attached to the patient. The signals are filtered (720), and the environment audio signal is then phase inverted (730). The phase-inverted environment signal is used to cancel (740) out non-breath sounds from the breath audio signal. The noise cancelled breath signal is then transformed (750) into the frequency domain. In many embodiments, a Fourier transform is used to transform into the frequency domain. In various embodiments, the Fourier transform is a fast Fourier transform (FFT), however other implementations can be used as appropriate to the requirements of specific applications of embodiments of the invention. In many embodiments, a wavelet transform is used instead. As can be readily appreciated, there are many different methods that can be used to transform from the time domain to the frequency domain. A spectrogram is then generated (760) using the magnitude vector from the transform. In various embodiments, the magnitude vector is linear and/or logarithmic. The spectrogram is provided (770) as input to a breath identification model. A breathing type identification is obtained (780) from the model and provided (790). In numerous embodiments, the identified breathing type is provided as an alert if the identified type is dangerous for the patient. Alerts can be audio alerts, visual alerts, tactile alerts, or any other alarm or combination thereof desired by the user. In many embodiments, a clinician may be automatically paged based on the alert.

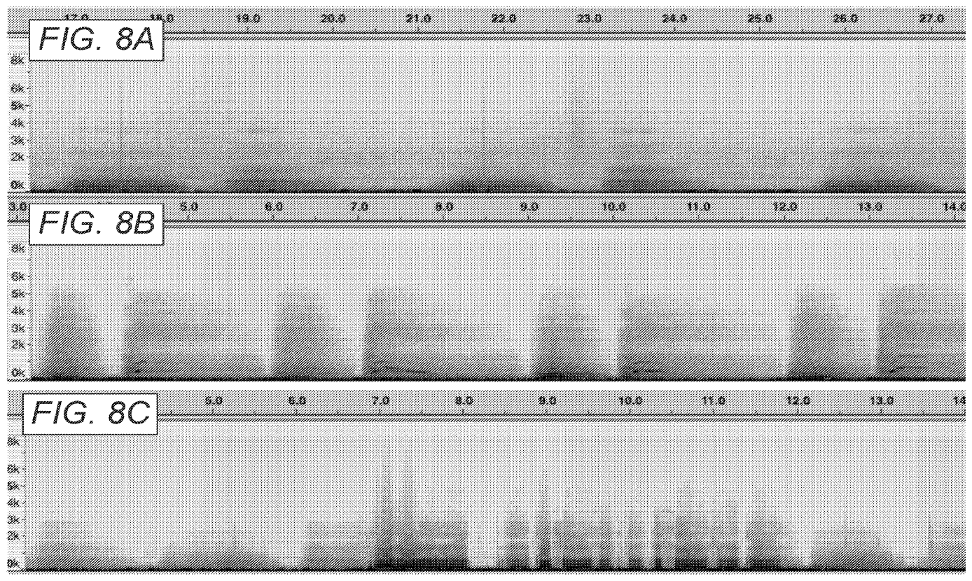

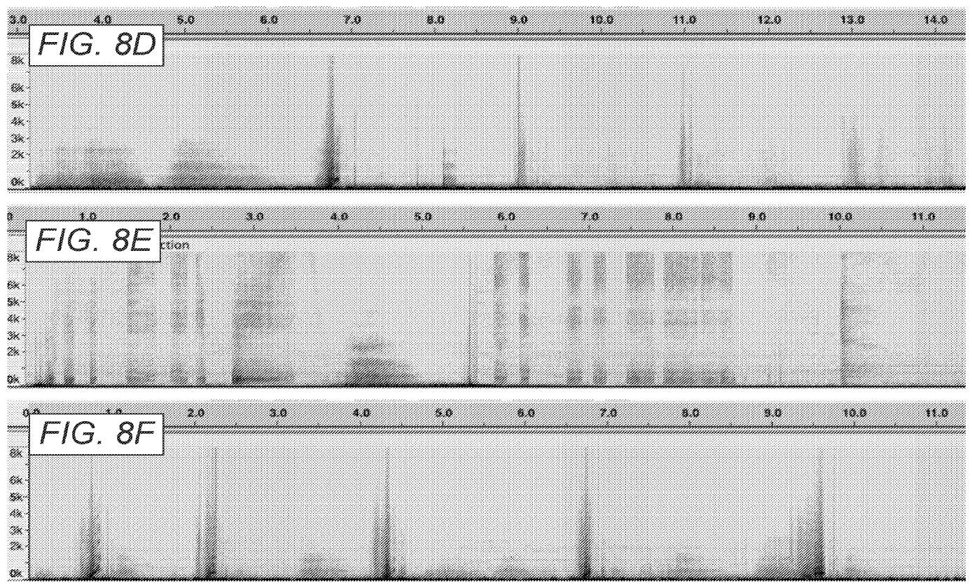

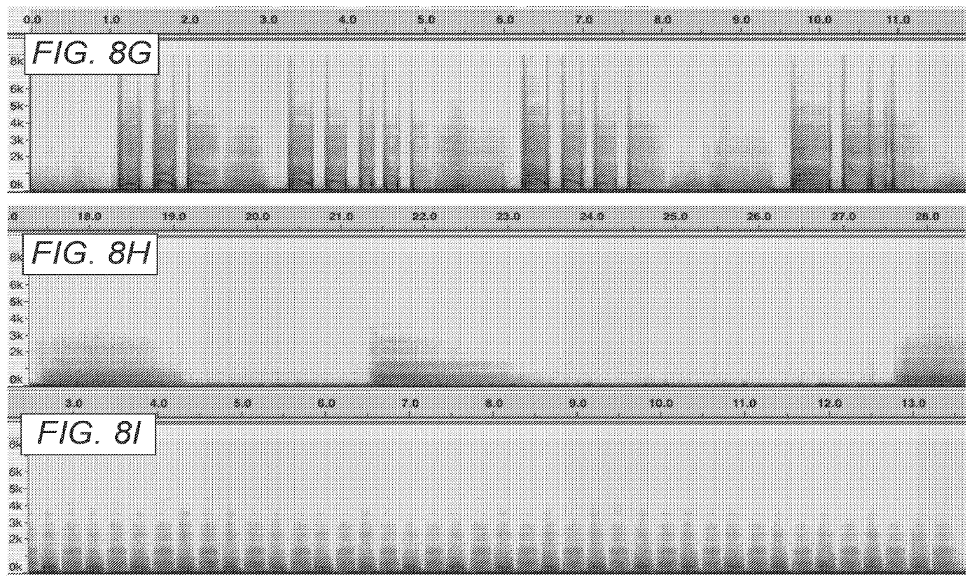

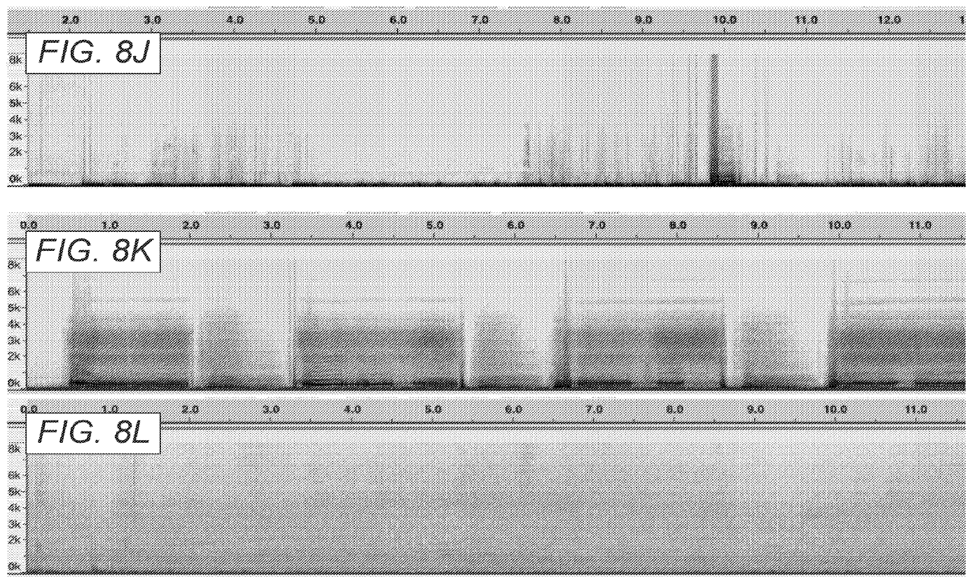

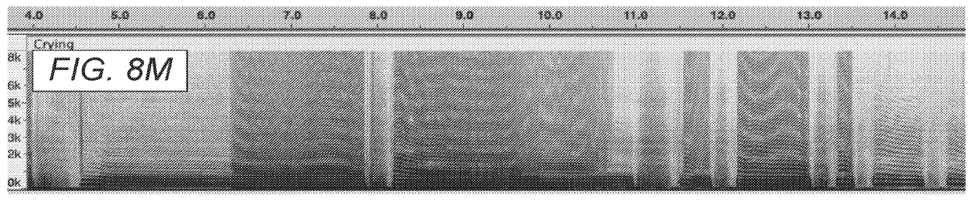

In numerous embodiments, the breathing identification model is a neural network. In various embodiments, the breathing identification model is a convolutional neural network. The model can be trained using a training data set made up of labeled spectrograms of breathing issues. In numerous embodiments, the training data is generated using listening devices as described herein. However, synthetic spectrograms or spectrograms obtained from other recording systems can be used as well. Any number of different breathing problems (and normal breathing) can be identified using the breathing model based on the training data. Further, in numerous embodiments, the model may return an “unidentified abnormal breathing” classification if the deviation from normal breathing is significant but does not resolve to a known breathing issue. FIGS. 8A-M illustrate spectrograms for various breathing issues based on signals recorded using listening devices as described herein, including: A) Normal Breathing; B) Expiratory Wheeze; C) Vocalization; D) Laryngospasm; E) Upper Airway Obstruction; F) Hiccups; G) Coughing; H) Bradypnea; I) Tachypnea; J) Swallowing; K) Inspiratory Stridor; L) Apnea; and M) Crying. However, other breathing problems can be identified as well, and the above examples are not provided as an absolute list of identifiable conditions and are merely provided as an example. As can be appreciated, any number of different breathing types, including those that are not necessarily deleterious, can be identified.

What is claimed is:

1. A respiration monitoring system, comprising:
a listening device, comprising:
a first microphone embedded in a gel matrix, where the gel matrix comprises a first channel between a surface of the gel matrix and the first microphone; and
a processing unit, comprising:
a processor; and
a memory, where the memory contains a respiration monitoring application that configures the processor to:
obtain a first audio signal from the first microphone, where the first audio signal comprises breath sounds recorded at the skin of a patient;
generate a spectrogram of the first audio signal;
provide the spectrogram to a breath identification model;
receive an identified breathing condition from the breath identification mode; and
provide the identified breathing condition.

2. The respiration monitoring system of claim 1, wherein:
the listening device further comprises a second microphone embedded in the gel matrix, where the gel matrix further comprises a second channel between the surface of the gel matrix and the second microphone, and where the first microphone and second microphone face opposite directions; and
the respiration monitoring application further configures the processor to:
obtain a second audio signal from the second microphone, where the second audio signal comprises audio signals from an environment of the patient;
phase invert the second audio signal;
apply noise cancellation to the first audio signal using the phase inverted second audio signal; and
generate the spectrogram using the noise cancelled first audio signal.

3. The respiration monitoring system of claim 2, wherein the first and second channels are filled with a material having a lower density than the gel matrix that allows sounds in the acoustic range of breathing to pass efficiently.

4. The respiration monitoring system of claim 2, further comprising a shell that covers a portion of the gel matrix over the second microphone.

5. The respiration monitoring system of claim 1, wherein to generate the spectrogram, the respiration monitoring application further directs the processor to generate a linear magnitude vector using a Fourier transform.

6. The respiration monitoring system of claim 1, wherein the listening device and the processing unit communicate wirelessly.

7. The respiration monitoring system of claim 1, further comprising a telemetry unit configured to receive the first audio signal and provide the first audio signal to the processing unit.

8. The respiration monitoring system of claim 1, further comprising a warning device configured to provide an alarm in response to an abnormal identified breathing condition.

9. The respiration monitoring system of claim 1, wherein the breath identification model is a convolutional neural network.

10. The respiration monitoring system of claim 1, wherein the respiration monitoring application further directs the processor to filter the first audio signal below 250 Hz and above 1500 Hz.

11. A respiration monitoring method, comprising:
obtaining, at a processing unit, a first audio signal from a first microphone, where:
the first audio signal comprises breath sounds recorded at the skin of a patient;
the first microphone embedded in a gel matrix; and
where the gel matrix comprises a first channel between a surface of the gel matrix and the first microphone;
generating a spectrogram of the first audio signal;
providing the spectrogram to a breath identification model;
receiving an identified breathing condition from the breath identification mode; and
providing the identified breathing condition.

12. The respiration monitoring method of claim 11, further comprising:
obtaining a second audio signal from a second microphone at the processing unit, where:
the second audio signal comprises audio signals from an environment of the patient;
the second microphone is embedded in the gel matrix;
the gel matrix further comprises a second channel between the surface of the gel matrix and the second microphone, and
the first microphone and second microphone face opposite directions;
phase inverting the second audio signal;
applying noise cancellation to the first audio signal using the phase inverted second audio signal; and
generating the spectrogram using the noise cancelled first audio signal.

13. The respiration monitoring method of claim 12, wherein the first and second channels are filled with a material having a lower density than the gel matrix that allows sounds in the acoustic range of breathing to pass efficiently.

14. The respiration monitoring method of claim 12, wherein the gel matrix is covered by a shell that over a portion of the gel matrix over the second microphone.

15. The respiration monitoring method of claim 11, wherein generating the spectrogram comprises generating a linear magnitude vector using a Fourier transform.

16. The respiration monitoring method of claim 11, wherein the first microphone and the processing unit communicate wirelessly.

17. The respiration monitoring method of claim 11, wherein the first microphone provides the first audio signal to a telemetry unit, which in turn provides the first audio signal to the processing unit.

18. The respiration monitoring method of claim 11, further comprising providing a warning using a warning device in response to an abnormal identified breathing condition.

19. The respiration monitoring method of claim 11, wherein the breath identification model is a convolutional neural network.

20. The respiration monitoring method of claim 11, further comprising filtering the first audio signal below 250 Hz and above 1500 Hz.
